(19) United States
(12) Plant Patent Application Publication
(10) Pub. No.: US 2009/0210984 P1
(43) Pub. Date: Aug. 20, 2009

(54) Blueberry plant named 'Lucero'

(50) ;

(75) Inventors: Antonio Abad Alamo, Almonte (ES); Jose Ulf Hayler Lopez, Almonte (ES); Paul M. Lyrene, Micanopy, FL (US)

Correspondence Address: BUCHANAN, INGERSOLL & ROONEY PC, POST OFFICE BOX 1404, ALEXANDRIA, VA, 22313-1404, US

(73) Assignee: ROYAL BERRIES S.L., Almonte (ES)

(21) Appl. No.: 12/289,493

(22) Filed: Oct. 29, 2008

(30) Foreign Application Priority Data
Feb. 14, 2008 (QZ) 2008/0340

(51) Int. Cl. A01H 5/00

(52) U.S. Cl. PLT/157

(57) ABSTRACT

A new and distinct Blueberry cultivar is provided that is the product of a controlled breeding program followed by selection. The cultivar flowers and forms fruit that ripens at mid-season. The attractive light blue substantially round berries are formed in compact clusters and exhibit a refreshing blueberry flavor. The berries are well amenable to mechanical harvest. The plant is self-fertile, and the growth habit is generally upright. Attractive evergreen foliage is displayed. A low chilling requirement is also exhibited.

### BOTANICAL/COMMERCIAL CLASSIFICATION

Vaccinium corymbosum L./Blueberry Plant

### VARIETAL DENOMINATION cv. Lucero

### SUMMARY OF THE INVENTION

The new Blueberry cultivar of the present invention was the product of controlled artificial pollination carried out in a greenhouse at Greenwood, Fla., U.S.A., wherein two parents were crossed which previously had been studied in the hope that they would contribute the desired characteristics. The female parent (i.e., the seed parent) was the unreleased ‘FL 91-160’ cultivar (non-patented in the United States). The male parent (i.e., pollen parent) was the ‘Windsor’ cultivar (U.S. Plant Pat. No. 12, 783). The parentage of the new cultivar can be summarized as follows:

‘FL 91-160’×‘Windsor’.

The seeds resulting from the pollination were shipped to Almonte, Huelva, Spain, where they sown during approximately 1997, small plants were obtained which were physically and biologically different from each other and selective research of the progeny was carried out. Selective study resulted in the identification of a single plant of the new cultivar. The new cultivar initially was designated S01-28-01.

It was found that the new Blueberry plant of the present invention displays the following combination of characteristics:
(a) flowers at mid-season and forms fruit that ripens at mid-season,
(b) displays a generally upright growth habit with attractive evergreen foliage,
(c) is self-fertile,
(d) displays a low chilling requirement, and
(e) forms in abundance attractive light blue substantially round berries having an extended post-harvest shelf life in compact clusters that exhibit a refreshing blueberry flavor and are well suited for mechanical harvest.

The new cultivar well meets the needs of the horticultural industry and can be grown to advantage for the commercial production of blueberries. The plant is well suited for growing under tunnels in well drained soils in warm regions while utilizing evergreen management techniques.

The new cultivar of the present invention can be distinguished from its ancestors and all other Blueberry cultivars known to its originators. When compared to the ‘Sharpblue’ cultivar (non-patented in the United States), the new cultivar is taller, more upright, and more resistant to Rust. When compared to the ‘Misty’ cultivar (non-patented in the United States), the new cultivar forms very compact berry clusters while the fruit of the ‘Misty’ cultivar commonly is borne in a considerably more expanded manner. When compared to the ‘O'Neal’ cultivar (non-patented in the United States), the new cultivar commonly displays a chill requirement of less than 300 hours, while the chill requirement of the ‘O'Neal’ cultivar commonly approximates 500 hours. When compared to the ‘Star’ cultivar (U.S. Plant Pat. No. 10,675), the new cultivar commonly is more resistant to Botrytis.

The new cultivar was selected during the spring of 2001 and has been asexually reproduced by the rooting of softwood cuttings beginning during the summer of 2001 at Almonte, Huelva, Spain. Such asexual propagation has shown that the characteristics of the new cultivar are firmly fixed and are stably transmitted from one generation to another. Accordingly, the new cultivar asexually reproduces in a true-to-type manner.

The new cultivar has been named ‘Lucero’.

### BRIEF DESCRIPTION OF THE PHOTOGRAPHS

The accompanying photographs show in color as nearly true as it is reasonably possible to make the same in color illustrations of this character, typical plants and plant parts of the new cultivar. The plants which had been asexually reproduced by the rooting of softwood cuttings were approximately five years of age except where otherwise indicated, and were being grown outdoors at Almonte, Huelva, Spain.

FIG. 1 shows a portion of an upright flowering plant of the new cultivar.

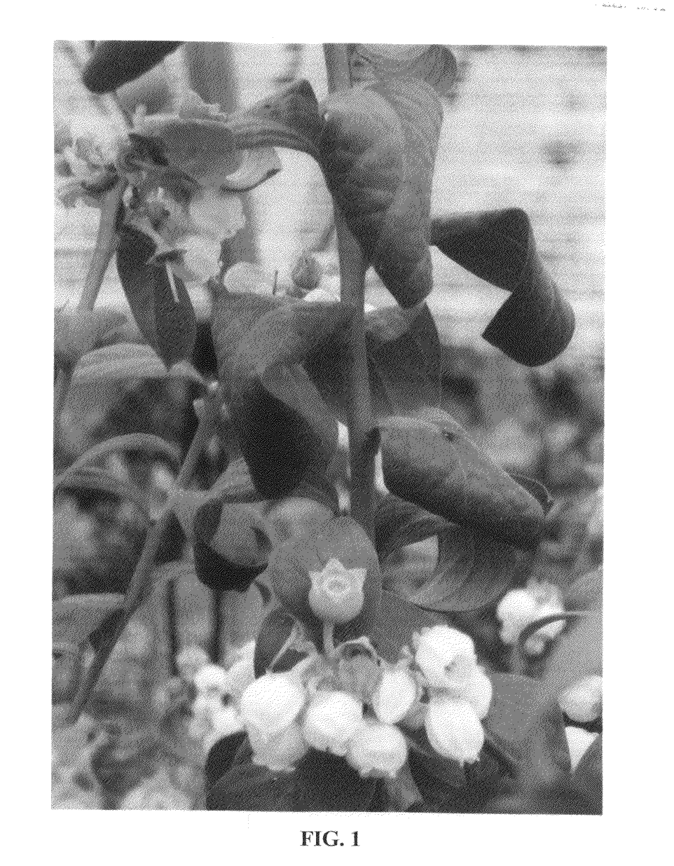

FIG. 2 shows a compact cluster of berries of the new cultivar in various stages of development as well as the foliage of the new cultivar.

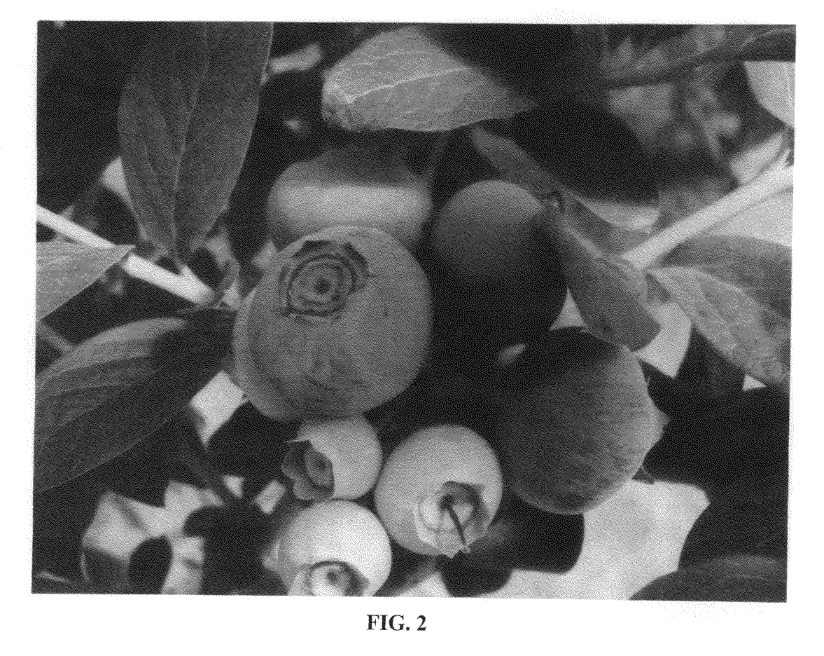

FIG. 3 shows a close view of the upper (adaxial)) surfaces of typical leaves of the new cultivar.

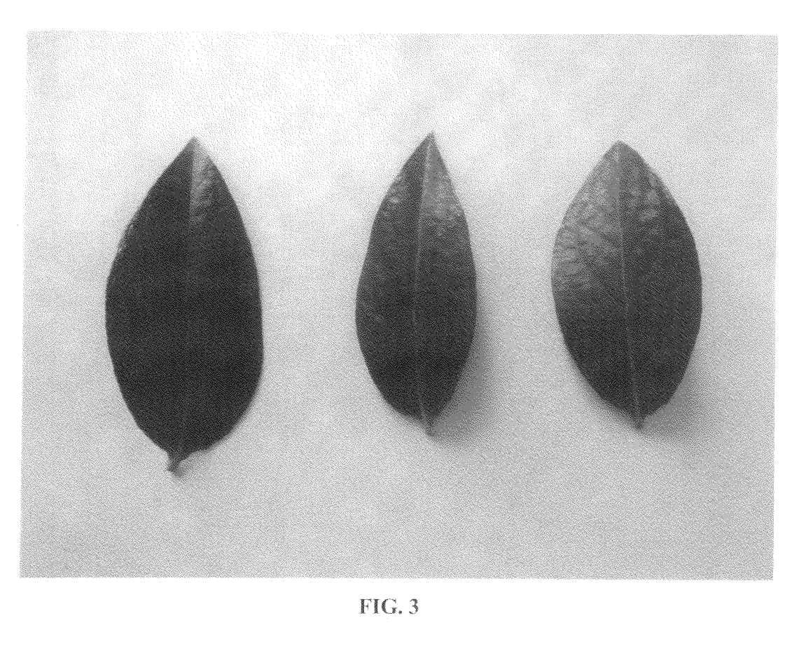

FIG. 4 shows a close view of the under (abaxial) surfaces of typical leaves of the new cultivar.

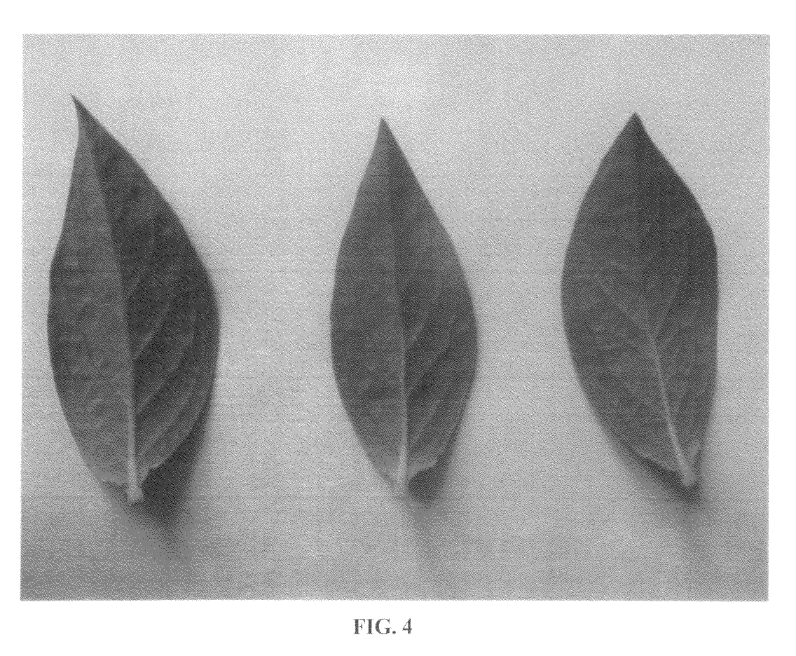

### DETAILED DESCRIPTION

The chart used in the identification of the colors described herein is the R.H.S. Colour Chart of The Royal Horticultural Society, London, England. Ordinary color terms are to be accorded their customary dictionary significance. The description is based on the observation of approximately five-year-old plants of the new cultivar which had been asexually reproduced by the rooting of softwood cuttings while growing outdoors at Almonte, Huelva, Spain.
Plant:
   Growth habit.—generally upright.
   Height.—approximately 1.75 m at 5 years of age.
   Width.—approximately 2.7 m at 5 years of age.
   Foliage retention.—evergreen.
   Chill requirement.—less than 300 hours.
Foliage:
   Shape.—generally elliptic (as illustrated).
   Length.—commonly approximately 59 mm on average.
   Width.—commonly approximately 27 mm on average.
   Apex.—acute.
   Base.—acute.
   Margin.—entire.
   Texture.—glabrous and non-glandular.
   Color.—Green Group 137B on the upper (adaxial) surface, and near Green Group 138C on the under (abaxial) surface.
Flowers:
   Time.—mid-season, at Almonte, Huelva, Spain, with first flower commonly at approximately January 20^{th}, and 50 percent bloom at approximately February 25^{th}.
   Number.—commonly approximately 6 flowers per bud on average.
   Petals.—5 in number and fused into a corolla tube.
   Fertility.—self-fertile.
   Fragrance.—none.
Fruit:
   Time.—commonly from approximately April 20th to June 15^{th} at Almonte, Huelva, Spain (i.e., approximately 8 weeks).
   Shape.—substantially round and somewhat pumpkin-shaped (as illustrated).
   Height.—commonly approximately 17 mm on average.
   Width.—commonly approximately 20 mm on average.
   Weight.—approximately 3.26 g/berry on average when plants were 5 years of age.
   Fruit scar.—approximately 1.4 mm in size, relatively deep, and dry.
   Seed number.—commonly approximately 28 per berry on average.
   Seed size.—commonly approximately 1.2 mm in length, and approximately 0.8 mm in width on average.
   Immature color.—commonly near Green Group 130D with bloom, and Yellow-Green Group 145A without bloom.
   Mature color.—light blue, Violet-Blue Group 97A with bloom, and Black Group 202A without bloom.
   Productivity.—abundant, approximately 3.21 Kg/plant on average when plants were 5 years of age.
   Flavor.—displays a refreshing blueberry flavor.
Development:
   Ability to store.—the fruit stores well under refrigeration, when stored at 8° C. approximately 100 percent of harvest commonly is good 7 days after harvest, and when stored at 20° C. approximately 96 percent of harvest commonly is good 7 days after harvest.
   Disease tolerance.—no special sensitivity to common Blueberry diseases, such as Leaf Rust (Pucciniastrum vaccini) and Botrytis (Botrytis cinerea), has been encountered during observations to date at Almonte, Huelva, Spain, and has shown to be more resistant to Rust than the “Sharpblue’ cultivar, and more resistant to Bortytis than the ‘Star’ cultivar.
   Insects.—is susceptible to aphids and thrips.
   Cultural conditions.—does well when grown in a warm low-chilling area under tunnels in well drained soils while using evergreen growing conditions.

Plants of the ‘Lucero’ cultivar have not been observed under all possible environmental conditions to date, Accordingly, it is possible that the phenotypic expression may vary somewhat with changes in light intensity and duration, cultural practices, and other environmental conditions without variance in the genotype.

We claim:

1. A new and distinct Blueberry plant that possess the following combination of characteristics:
(a) flowers at mid-season and forms fruit that ripens at mid-season,
(b) displays a generally upright growth habit with attractive evergreen foliage,
(c) is self-fertile,
(d) displays a low chilling requirement, and
(e) forms in abundance attractive light blue substantially round berries having an extended post-harvest shelf life in compact clusters that are well suited for mechanical harvest and exhibit a refreshing blueberry flavor;
substantially as herein shown and described.
